(19) United States
(12) Patent Application Publication
(10) Pub. No.: US 2012/0082736 A1
(43) Pub. Date: Apr. 5, 2012

(54) Small-molecule TNF modulator to reduce the side effects of chemotherapy and radiotherapy

(75) Inventor: Jiajiu SHAW, Ann Arbor, MI (US)

(73) Assignees: GeneBlue Corporation, San Gabriel, CA (US); 21st Century Therapeutics, Inc., Ferndale, MI (US)

(21) Appl. No.: 13/244,594

(22) Filed: Sep. 25, 2011

Related U.S. Application Data
(60) Provisional application No. 61/389,131, filed on Oct. 1, 2010.

(51) Int. Cl. A61K 31/42; A61K 31/282; A61P 35/00; A61P 35/02; A61P 1/16; A61P 7/00; A61P 7/02; A61K 33/24; A61P 13/12

(52) U.S. Cl. 424/649; 514/378; 514/492

(57) ABSTRACT

Cancer patients treated by chemotherapy and/or radiotherapy often suffer serious side effects. Currently, there is only one FDA approved and used as both a chemoprotector and a radioprotector, amifostine, which is associated with significant problems. Disclosed in the present invention are novel methods of using UTL-5g as both a chemoprotector and radioprotector for treating cancer patients in addition to other related methods.

### RELATED APPLICATION

The present invention is based on U.S. Provisional Application Ser. No. 61/389,131, filed on Oct. 1, 2010 to which priority is claimed under 35 U.S.C 120 and of which the entire specification is hereby expressly incorporated by reference.

### TECHNICAL FIELD

Chemoprotection and Radioprotection

### BACKGROUND

Chemotherapy, Cisplatin, and Amifostine

Cisplatin, cis-dichlorodiammine platinum, is a widely used cytotoxic agent with therapeutic activity against various tumors, but also with substantial side effects [Rosenberg, 1979]. The clinical use of cisplatin is mainly limited by its side effects such as nephrotoxicity (the major side effect) which evolves slowly but predictably after initial and repeated exposure [Madias, 1978; Ries, 1986; Zhang, 2007]. Among the earliest reactions of the kidney is the activation of the mitogen-activated protein kinase (MAPK) cascade and molecular events typical of the stress responses. Metabolic responses and the inflammatory cascade are important determinants of the degree of renal failure induced by cisplatin. Manipulation of these stress responses may be exploited to reduce the toxicity of cisplatin clinically. Since TNF-α plays an important role in modulating these metabolic events, it is a legitimate biological target of chemoprotection. Another side effect of cisplatin is hepatotoxicity which is the dose-limiting toxicity. Hepatotoxicity occurs when cisplatin is administered at high doses [Pollera, 1987]. Yet another side effect of cisplatin is myelosuppression [Gill, 1991; Kelland, 1999], which occurs in 25 to 30 percent of patients who undergo treatments with cisplatin. Myelosuppression relates to a reduction of activity in the bone marrow, in particular, toxicity to the blood forming elements. The levels of platelets and white blood cells (or leukocytes) associated with myelosuppression are generally lowest about 3 weeks after treatment and return to normal a little more than 2 weeks thereafter. The loss of platelets (thrombocytopenia) and the loss of leukocytes (leukopenia or leukocytopenia) are more pronounced when higher doses of cisplatin are given. In addition to the loss of platelets and white blood cells, the use of cisplatin can also cause a decrease in the number of red blood cells (anemia).

A chemoprotective agent which reduces the side effects of cisplatin described above without affecting it therapeutic effect would have significant clinical benefit. Although a number of natural and synthetic compounds have been shown to be chemoprotective [Subbiah 2008; Li, 1995; Psotova, 2004], the only FDA approved and generally accepted chemoprotective drug for cisplatin therapy is amifostine (Ethyol®), which is a sulfur-containing agent that reduces toxicity to various chemotherapy and radiotherapy regimens [Korst, 1996; Markman 1998; Phillips, 1998]. It has some chemoprotective effects against cisplatin-related renal toxicity and neutropenia due to cisplatin-cyclophosphamide combination therapy.

Radiotherapy, Radioprotection, and Amifostine

Radiotherapy is usually used to treat almost every type of solid tumor, including cancers of the breast, brain, lung, cervix, pancreas, prostate, skin, stomach, and uterus. It can also be used to treat leukemia (cancers of blood-forming cells) and lymphoma (cancer of the lymphatic system) respectively. Radiotherapy has been used for curative or adjuvant cancer treatment (for example, radiotherapy is usually given after surgery or in conjunction with chemotherapy) for many years.

Radiation therapy works by damaging the DNA of tumor cells to hamper these cells from growing/replicating. The damage is caused by the high energy beam directly or indirectly ionizing the atoms which make up the DNA chain; most of the therapeutic effect is resulted from free radicals produced by radiation. Because cancer cells are generally under-differentiated, they tend to have a lower ability to repair DNA damages. As a result, the DNA damage in cancer cells causes them to die or significantly reduces their reproduction.

Although normal cells are fully differentiated and generally more capable to repair certain DNA damage, they are still subject to radiation damage. In addition to the DNA damage onto normal cells by radiotherapy, there are additional undesirable biological impacts on the body, including abnormal immunological responses (such as elevation of cytokines, including TNF-α) and potential damage to bone marrow.

Many approaches have been made by the medical community to protect normal tissues or ameliorate tissue injury induced by radiotherapy. In general, there are three major approaches: (1) improvements on technical aspects of radiotherapy, (2) use of radiosensitizers to enhance the killing of radio-resistant cancer cells, and (3) use of radioprotective agents to protect normal tissues and/or reduce the side effects. As of today, there is only one FDA approved drug that's been used for both chemoprotection and radioprotection, amifostine.

Problems with Amifostine

While protecting normal cells, amifostine has also been reported to actually protect tumor cells in some animal studies [Ethyol, 1996]. In addition, amifostine has a number of undesirable side effects including hypotention, diarrhea, nausea, hypocalcemia, etc. Therefore, there is ample room for improvement on amifostine.

Although amifostine is an FDA approved radioprotector/chemoprotector, there are significant limitations associated with amifostine including:
1. The major limitation of amifostine is that simultaneous administration of a sulfur-containing chemoprotector with cisplatin has shown rapid chemical quenching of cisplatin by the sulfur anion or the sulfhydryl moiety to form an inactive platinum-thiol conjugate species [Hausheer, 1998]. Animal data suggest that amifostine may have tumor-protective effect and thus amifostine is not recommended with curative chemotherapy outside of a clinical trial [Ethyol, 1996; Ethyol, 1998].
2. Amifostine is associated with certain side-effects including nausea and vomiting, as well as transient hypotension [Mabro, 1999]. While the use of amifostine is becoming more widespread, an increased number of cutaneous reactions have also been reported [Demiral, 2002].
3. Because amifostine and its metabolite both have very short half-lives and are rapidly cleared from the plasma (95% and 50% of the peak concentration within 1 h, respectively) [Korst, 1996], amifostine is usually administered by a 15 minute infusion 30 minutes before cisplatin chemotherapy, which is inconvenient [Ethyol, 2010].

Therefore, it is of great importance and interest to develop a new agent that is both chemoprotective and radioprotective; such agent has a better efficacy and/or a lower toxicity as compared to amifostine.

### SUMMARY

### Technical Problem

Cancer patients treated by chemotherapy and/or radiotherapy often suffer serious side effects. Currently, there is only one FDA approved and used as both a chemoprotector and a radioprotector, amifostine, which is associated with significant problems as described in the previous sections.

### Solution to Problem

Disclosed is a small molecule TNF-α inhibitor, UTL-5g, which works as both a chemoprotector and a radioprotector.

### Advantageous Effects of Invention

UTL-5g shows superior chemoprotection effect as compared to amifostine. Surprisingly, UTL-5g increased the efficacy of cisplatin whole reducing the side effects of cisplatin. UTL-5g also shows significant radioprotective effect. In addition, UTL-5g has a significantly lower acute toxicity as compared to amifostine.

### BRIEF DESCRIPTION OF DRAWINGS

FIG. 1. BUN and AST induced by cisplatin in 15 days and in 7 days (a) First study (day 0, 2, 4, 7, 9, 11, and 15). (b) Second study (day 0, 5, 6, and 7). Each data point represents the average of 2 mice.

FIG. 2. Effect of cisplatin on BUN (a) and creatinine (c); effect of UTL-5g pretreatment on BUN (b) and creatinine (d) in mouse treated with cisplatin

Gp 1: medium (saline). Gp 2: saline followed by cisplatin (2.5 mg/kg) 30 min later. Gp 3: UTL-5g (15 mg/kg) followed by cisplatin (2.5 mg/kg) 30 min later. Gp 4: UTL-5g (30 mg/kg) followed by cisplatin (2.5 mg/kg) 30 min later. Gp 5: UTL-5g (60 mg/kg) followed by cisplatin (2.5 mg/kg) 30 min later. Gp 6: amifostine (200 mg/kg) followed by cisplatin (2.5 mg/kg) 30 min later; Gp 7: saline followed by cisplatin (5 mg/kg) 30 min later. Gp 1-Gp 6 were daily ×5, but Gp 7 was by single dose on day 0 only. StDev is shown for each Gp (n=5 for each group except for Gp 7, n=3). ** two-tailed p<0.005 vs medium control by Student's t-test.

FIG. 3. Effect of cisplatin on AST (a) and ALT (c); effect of UTL-5g/amifostine pretreatment on AST (b) and ALT (d) for mice treated with cisplatin

Gp 1: medium (saline). Gp 2: saline followed by cisplatin (2.5 mg/kg) 30 min later. Gp 3: UTL-5g (15 mg/kg) followed by cisplatin 30 min later. Gp 4: UTL-5g (30 mg/kg) followed by cisplatin (2.5 mg/kg) 30 min later. Gp 5: UTL-5g (60 mg/kg) followed by cisplatin (2.5 mg/kg) 30 min later. Gp 6: amifostine (200 mg/kg) followed by cisplatin (2.5 mg/kg) 30 min later. Gp 7: saline followed by cisplatin (5 mg/kg) 30 min later. Gp 1-Gp 6 were daily ×5, but Gp 7 was by single dose on day 0 only. StDev is shown for each Gp (n=5 for each group except n=4 for Gp 7). * P<0.05, ** p<0.005 vs. medium control (Gp 1) by Student's t-test (two-tailed).

FIG. 4. Effect of UTL-5g/amifostine pretreatment on (a) WBC count, and (b) platelet counts for mice treated with cisplatin.

Gp 1: medium (saline). Gp 2: saline followed by cisplatin (2.5 mg/kg) 30 min later. Gp 3: UTL-5g (15 mg/kg) followed by cisplatin 30 min later. Gp 4: UTL-5g (30 mg/kg) followed by cisplatin (2.5 mg/kg) 30 min later. Gp 5: UTL-5g (60 mg/kg) followed by cisplatin (2.5 mg/kg) 30 min later. Gp 6: amifostine (200 mg/kg) followed by cisplatin (2.5 mg/kg) 30 min later. Gp 7: saline followed by cisplatin (5 mg/kg) 30 min later. Gp 1-Gp 6 were daily ×5, but Gp 7 was by single dose on day 0 only. StDev is shown for each Gp (n=5 for each group except n=4 for Gp 7). * P<0.05, ** p<0.005, *** p<0.0005 vs. medium control by Student's t-test (two-tailed).

FIG. 5. Effect of UTL-5g on TNF-α levels in plasma for mice treated with cisplatin. Gp 1, medium control (saline) only; Gp 2, saline followed by cisplatin (2.5 mg/kg) 30 min later; Gp 3, UTL-5g (15 mg/kg) followed by cisplatin 30 min later; Gp 4, UTL-5g (30 mg/kg) followed by cisplatin 30 min later; Gp 5, UTL-5g (60 mg/kg) followed by cisplatin 30 min later. Y axis represents the relative levels of TNF-α assuming 0% for Gp 1 (Control). Level of TNF-α on the figure is based on the average of 2 readings.

FIG. 6. Effect of UTL-5g pretreatment on (a) WBC, and (b) platelet counts for mice treated with cisplatin

Gp 1: medium (saline). Gp 2: saline followed by cisplatin (2.5 mg/kg) 30 min later. Gp 3: UTL-5g (15 mg/kg) followed by cisplatin 30 min later. Gp 4: UTL-5g (30 mg/kg) followed by cisplatin (2.5 mg/kg) 30 min later. Gp 5: UTL-5g (60 mg/kg) followed by cisplatin (2.5 mg/kg) 30 min later. Gp 6: amifostine (200 mg/kg) followed by cisplatin (2.5 mg/kg) 30 min later. Gp 7: saline followed by cisplatin (5 mg/kg) 30 min later. Gp 8: UTL-5g (60 mg/kg) followed by medium (saline) 30 min later. Gp 1-Gp 6 were daily ×5, but Gp 7 was by single dose on day 0 only. StDev is shown for each Gp (n=5 for each group except n=4 for Gp 7). * P<0.05, ** p<0.005 vs. medium control by Student's t-t (two-tailed).

FIG. 7. Effect of UTL-5g pre-treatment on the antitumor effect of cisplatin Group 1, saline, daily ×5 (control); Group 2, cisplatin (2.5 mg/kg) in saline, daily ×5; Group 3, UTL-5g (60 mg/kg) followed by cisplatin (2.5 mg/kg) 30 min later, daily ×5. Two tailed t test shows p<0.01 for Control vs. cisplatin; p=0.01 for Group 1 vs. Group 5; p=0.03, for Group 4 vs. Group 5 (UTL-5g+cisplatin), n=5. Gp: Group

FIG. 8. Effect of UTL compounds on AST and ALT with and without radiation C57BL/6 male mice were treated with UTL-5b, -5d, and -5g each in 0.3 mL vehicle containing DMSO/(Cremophor:propylene glycol 4:6 v/v)/saline, 5/5/90 v/v by i.p. injection (30 mg/kg) 1 hr prior to liver irradiation with 15 Gy. Serum was obtained at 2 hr after irradiation and assayed for AST and ALT activities. Data are means±standard deviation (S.D.) * indicates p<0.05, n=4.

FIG. 9. Dose dependent radioprotection of liver by UTL-5g

Mice were treated with various doses of UTL-5g (3.75, 7.5, 15, 30, and 60 mg/kg, i.p.) 1 hr prior to liver radiation with 15 Gy. Serum was obtained at 2 hr after irradiation and assayed for AST (4a) and ALT activities (4b). Data shown are means±standard deviation (S.D.) *Significantly reduced AST or ALT levels as compared to irradiated mice treated with vehicle only, p<0.05 (n=4). Two-tailed paired t-Test showed p<0.05.

FIG. 10. Dose-dependent reduction of TNF-α

Mice were treated individually with 0.3 mL each of UTL-5g preparations i.p. 1 hr prior to liver irradiation at 15 Gy. Liver TNF-α levels were analyzed by standard ELISA methodology. TNF-α levels in irradiated liver (at 15 Gy) were increased by 56% (from 37.6 to 58.9 pg/n) and the pretreatment by UTL-5g significantly reduced TNF-α by 20% and 29% at 30 mg/kg and 60 mg/kg, respectively (* p<0.05, 2-tailed paired t-Test comparing treated and untreated for both groups, n=4).

FIG. 11. Radiation dose escalation and radioprotection by UTL-5 g Mice were pre-treated with UTL-5g (60mg/kg, i.p.) at 1 hr prior to radiation dose escalation (0, 5, 15, and 25 Gy). Serum was harvested at 2 hr after injections and was subjected to AST/ALT measurements as described in Methods. Results were compared to control mice received control vehicle. Data are means±standard deviation (* p<0.05, 2-tailed paired t-Test comparing treated and untreated groups, n=4).

FIG. 12. UTL-5g reduced radiation-induced liver apoptosis measured by TUNEL (Terminal deoxynucleotidyl transferase dUTP nick end labeling) staining

Mice were pre-treated with various doses of UTL-5g (7.5, 15, 30, and 60 mg/kg, i.p.) or control vehicle at 1 hr prior to liver radiation with 15 Gy. Liver tissues were harvested at 2 hr after radiation for cryosections and were subjected to TUNEL staining Positive cells were counted under fluorescent microscope (40×) from 5 randomly selected fields and were plotted as averages. *TUNEL(+) cell numbers were significantly reduced relative to vehicle (p<0.05, n=4) (6A). Representative liver sections showing TUNEL positive cells (green) counter-stained with DIPA (4′-6-diamidino-2-phenylindole) for nuclei under fluorescent microscope (40×) (6B). RT: radiation treatment.

### DESCRIPTION OF EMBODIMENTS

Recently, we investigated a novel small-molecule TNF-α modulator, UTL-5g (also known as GBL-5g), as an improved chemoprotective agent over amifostine to reduce the toxicity of cisplatin therapy to normal cells without compromising its cancer killing activity. Results of our studies are surprisingly positive, including: (1) UTL-5g lowered the elevated levels of blood urea nitrogen (BUN), creatinine, aspartate transaminase (AST), and alanine transaminase (ALT) induced by cisplatin; (2) UTL-5g lowered the elevated TNF-α levels induced by cisplatin in a dose-dependent manner; (3) UTL-5g did not reduce the therapeutic effect of cisplatin; it actually increased the therapeutic effect of cisplatin under current experimental condition. These animal study results, especially the enhancement of the therapeutic effect of cisplatin, are novel and scientifically significant.

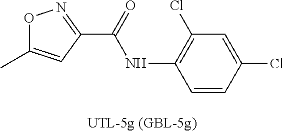

We also investigated the effect of UTL-5g in radioprotection and surprisingly significant radioprotective effect by UTL-5g was observed.

### REPRESENTATIVE EXAMPLES OF CHEMOPROTECTION BY UTL-5G

### Example 1

### To Determine a Suitable Day to Sacrifice the Animals Treated by Cisplatin

In order to see the maximum side effects (BUN and AST) so that the reduction of the side effects can be more easily observed, this animal study was conducted to find a suitable day to sacrifice the animals.

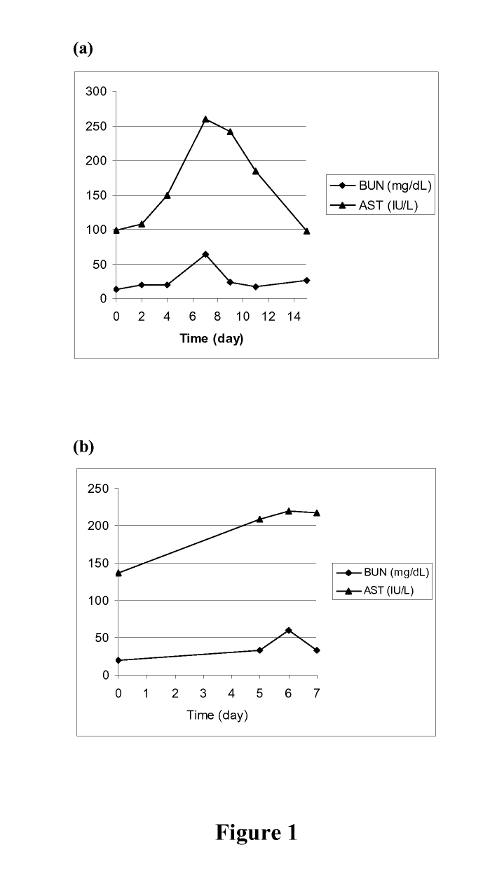

First, 0.25 mL of 0.2 mg/mL cisplatin (eq to 2.5 mg/kg) in saline was injected ip, daily ×5, in BDF1 mice. Saline was used as a control. Two mice were used per group and sacrificed on day 0 (control), 2, 4, 7, 9, 11, and 15. In addition, 2 BDF1 mice were injected with a higher dose, 0.25 mL of 0.4 mg/mL cisplatin (eq to 5 mg/kg), daily ×5. Unfortunately, for the higher dose, 1 mouse died on day 7 and the second was euthanized on day 14. This confirms the MTD of cisplatin was <5 mg/kg by ip daily ×5 as shown in the MTD study. Blood analysis results from the 2.5 mg/kg study indicated that the optimal ay to sacrifice the animals was around day 7 (FIG. 1a).

To further narrow down the exact day between day 4 and 7, a follow-up animal study was conducted employing cisplatin at 2.5 mg/kg and the mice were sacrificed on day 0 (control), 5, 6, and 7 (2 mice per point). The results (FIG. 1b) indicated that day 6 is the optimal day to sacrifice the animals in order to see the maximum side induced by cisplatin under current experimental condition.

### Example 2

### Effects of UTL-5g on BUN/Creatinine, AST/ALT, WBC/Platelet and TNF-α in Blood

This animal study was designed to show the effect of UTL-5g on BUN/creatinine, AST/ALT, and WBC/platelet counts to correlate with functions of kidney, liver, and bone marrow individually.

BDF1 female mice (average wt ˜20 g/mouse) were randomly divided into the following groups (5 mice per group) and each treated daily ×5 (starting from day 0) by ip injection (0.25 mL/mouse), except in Gp 7, each was treated by a single dose ip injection (0.25 mL/mouse) on day 0 only, as described below
Gp 1: medium (saline) followed by medium (saline) 30 min later
Gp 2: medium (saline) followed by cisplatin (2.5 mg/kg) 30 min later
Gp 3: UTL-5g (15 mg/kg) followed by cisplatin (2.5 mg/kg) 30 min later
Gp 4: UTL-5g (30 mg/kg) followed by cisplatin (2.5 mg/kg) 30 min later
Gp 5: UTL-5g (60 mg/kg) followed by cisplatin (2.5 mg/kg) 30 min later
Gp 6: amifostine (200 mg/kg) followed by cisplatin (2.5 mg/kg) 30 min later
Gp 7: medium (saline) followed by cisplatin (5 mg/kg) 30 min later (single dose on Day 0 only)

Preparation of the test samples are described below:
Cisplatin: Cisplatin was dissolved in saline (0.2 and 0.4 mg/mL in saline) for 2.5 and 5 mg/kg ip injection.
UTL-5g: The stock solution of UTL-5g was first dissolved in DMSO and equal volume of Cremophor/propylene glycol (60/40 v/v) was added to make a stock solution. Immediately before the injection, saline was added to this temporary solution at a 90:10 v/v ratio and mixed thoroughly. Three concentrations for ip injection were prepared (1.25, 2.5, and 5 mg/mL) so that the doses are equivalent to 15, 30, and 60 mg/kg with a 0.25 mL injection.
Amifostine: The stock solution of amifostine was dissolved in saline to the desired concentration (16 mg/mL) for the 200 mg/kg injection.

Cardiac puncture was used to obtain blood from each mouse on day 6. To assess liver/kidney protection by UTL-5g, all blood samples were analyzed for BUN, creatinine, AST, and ALT by the chemistry lab at Henry Ford Health System. Unopette® (Becton Dickinson, Franklin Lakes, N.J.) diluting systems and a hemocytometer were used to assess the effect of UTL-5g on WBC and platelet counts in blood.

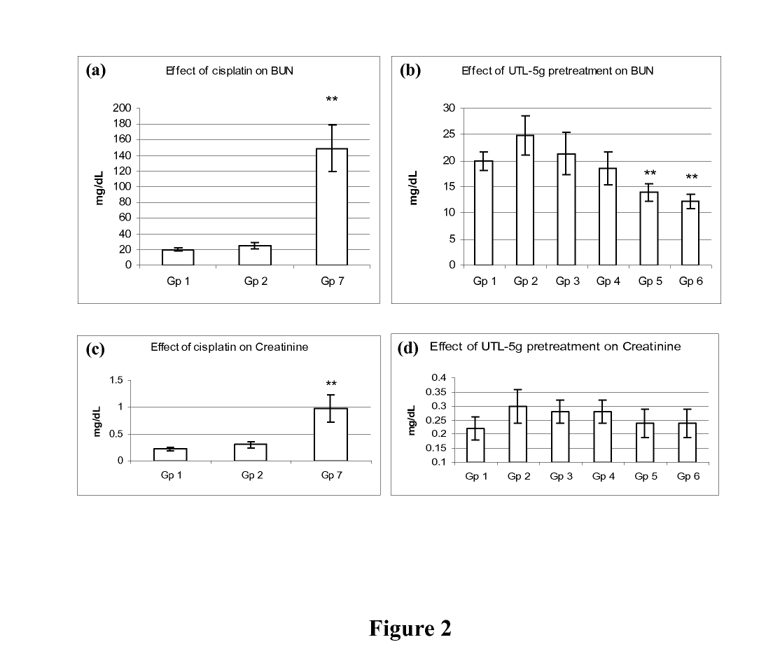

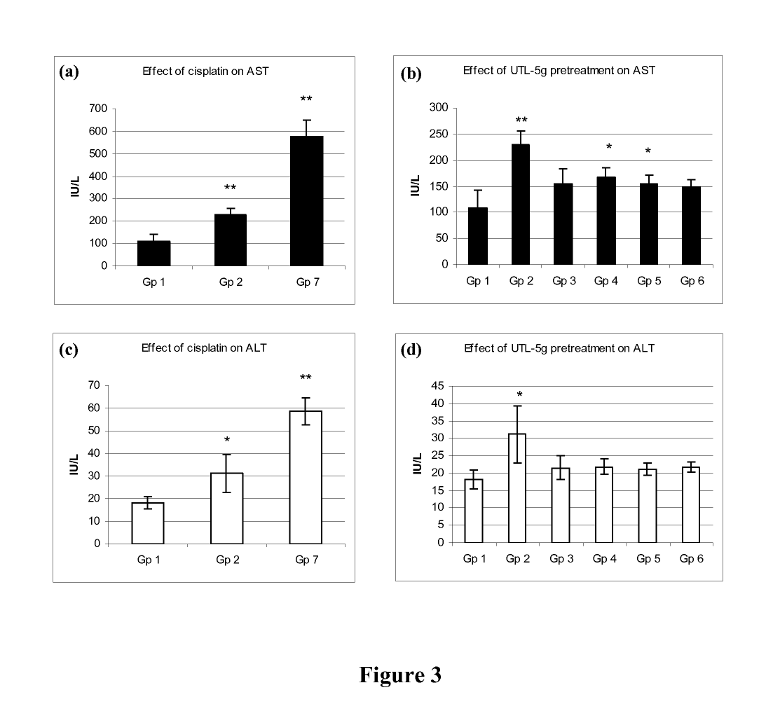

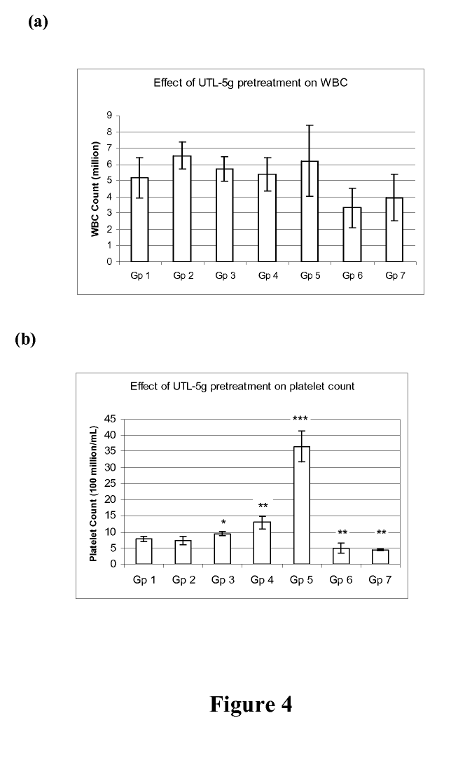

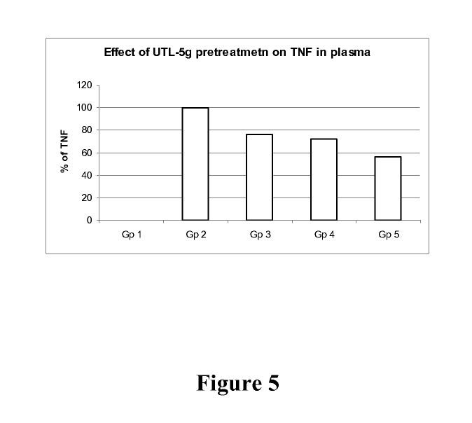

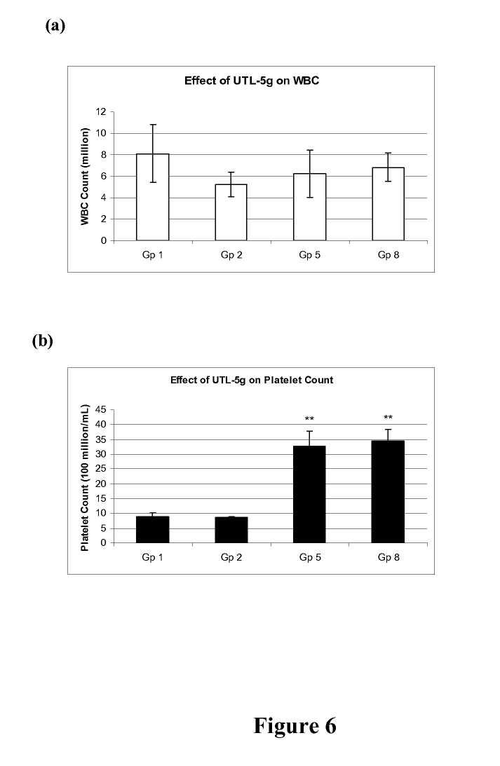

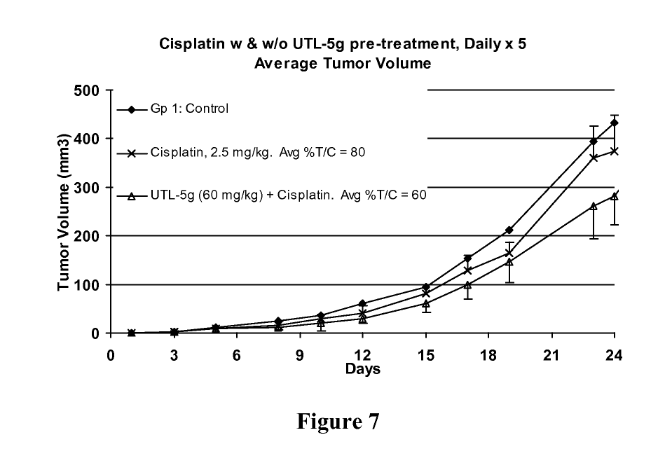

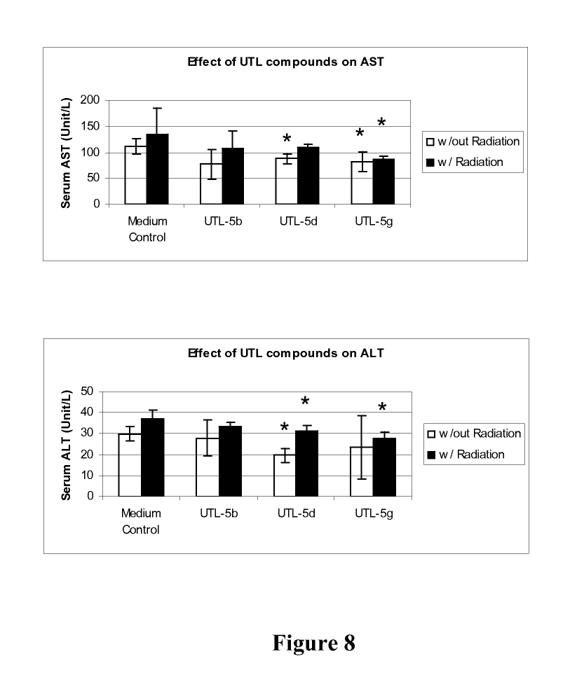

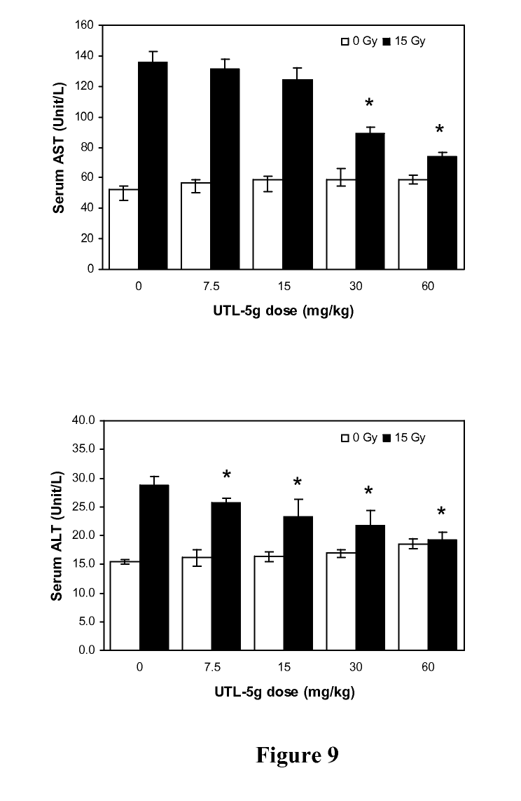

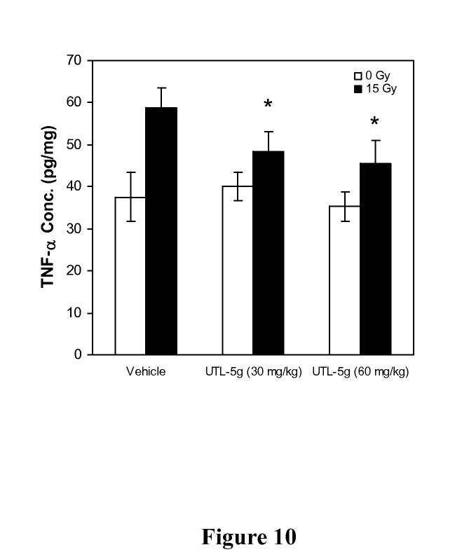

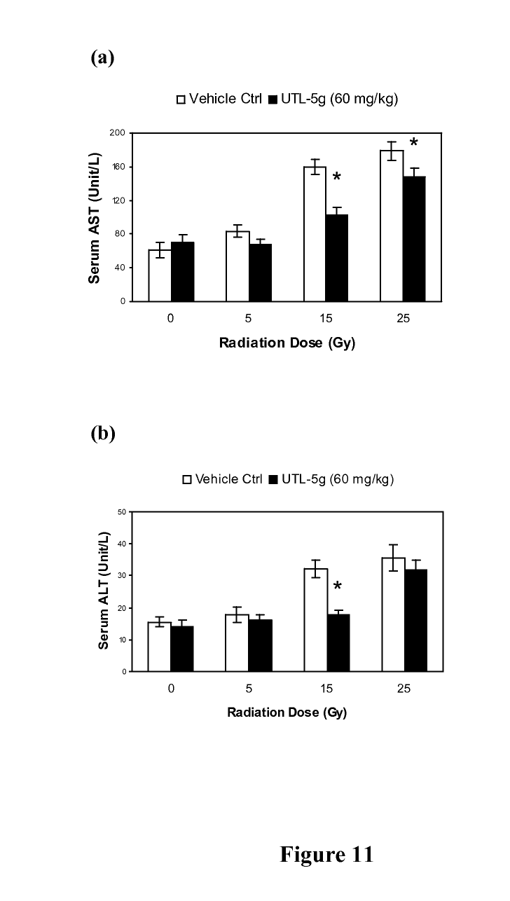

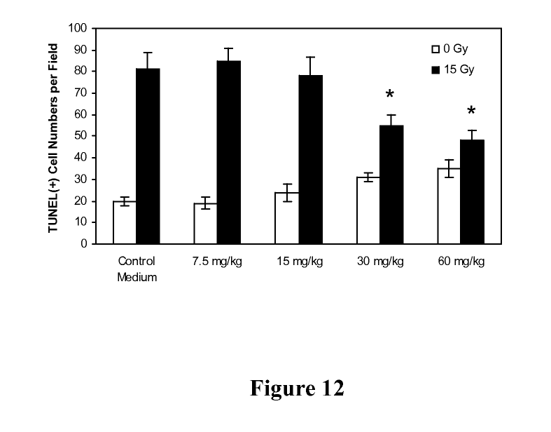

As shown in FIG. 2a, BUN levels were elevated by cisplatin in a dose-dependent manner. FIG. 2b shows that levels of BUN were suppressed by UTL-5g in a dose-dependent manner. The optimal dose of UTL-5g (60 mg (or 0.22 mmole)/kg) showed essentially the same reduction (Gp 5) as compared to that from amifostine at a much higher dose of 200 mg (or 0.93 mmole)/kg (Gp 6). Essentially the same results were obtained for creatinine (FIGS. 2c and 2d). The reduction of BUN and creatinine by UTL-5g indicate that kidney damage induced by cisplatin may be reduced by the pretreatment of UTL-5g.

As shown in FIGS. 3a and 3b, AST levels were elevated by cisplatin and reduced by UTL-5g. FIGS. 3c and 3d show that ALT levels were elevated by cisplatin and also reduced by UTL-5g. Again, UTL-5g reduced the elevated AST and ALT as compared to amifostine. The results indicate that UTL-5g may reduce the liver damage induced by cisplatin in vivo.

As shown in FIG. 4a, pretreatment of UTL-5g, for mice treated with cisplatin at 2.5 mg/kg by ip injection, daily ×5, did not significantly increase the WBC count. This may be partially due to the low dose of 2.5 mg/kg since higher dose of cisplatin (Gp 7) does show lower WBC count as compared to Control (Gp 1).

As to the platelets, pretreatment of UTL-5g, for mice treated with cisplatin, increased platelet count in a dose-dependent manner (FIG. 4b) and the effect is especially profound for 60 mg/kg (>3 times of the control). Comparing Gp 7 and 1 (Control), it is obvious that cisplatin, at 5 mg/kg, did decrease platelet count significantly. Comparing Gp 3, 4, and 5 with Gp 1 (Control), the increase of platelet count is dose-dependent and statistically significant. On the contrary, the pretreatment of amifostine (Gp 6) as compared to Gp 1 (Control) shows a statistically significant decrease on platelet count.

Using the samples from the same animal study, TNF-α levels in plasma were analyzed. A commercial assay kit was used and the testing was conducted according to the procedure provided by the manufacturer (eBioscience). The results indicated that TNF-α was elevated by cisplatin as expected and the pretreatment of UTL-5g lowered elevated TNF-α in plasma in a dose-dependent manner as shown in FIG. 5. Because most blood from each mouse was used for the blood analysis (shown in previous section) and insufficient amount of blood was available for individual test, the plasma for each group was obtained from pooled blood from individual mice in each group.

### Example 3

### Does UTL-5g Increase Platelet Count by Itself?

To further investigate whether UTL-5g by itself would increase platelet count, the following small add-on study was conducted:
Gp 1: medium (saline) followed by medium (saline) 30 min later (same as Gp 1 before)
Gp 2: medium (saline) followed by cisplatin (2.5 mg/kg) 30 min later (same as Gp 2 before)
Gp 5: UTL-5g (60 mg/kg) followed by cisplatin (2.5 mg/kg) 30 min later (Same as Gp 5 before)
Gp 8: UTL-5g alone (60 mg/kg) followed by medium (saline) 30 min later (New Gp)

Results from this animal study are shown in FIG. 6. Again, no significant protective effect of UTL-5g on WBC was observed as shown in FIG. 6a. However, it was obvious that UTL-5g significantly increased platelet count whether the mice were treated by cisplatin (2.5 mg/kg) later or not (FIG. 6b). The results confirm that pretreatment of UTL-5g increased the platelet count that was reduced by cisplatin. In addition, UTL-5g by itself stimulated the production of platelets.

### Example 4

### To Demonstrate that UTL-5 g Does Not Decrease Cancer Killing Effect of Cisplatin

In order to qualify UTL-5g as a chemoprotective agent, a therapeutic assessment was conducted to evaluate the effect of UTL-5g on the efficacy of cisplatin. Briefly, 25 SCID mice were randomly divided into 5 groups as below.
Gp 1: Saline daily ×5 (control)
Gp 2: Cisplatin 0.2 mg/mL (2.5 mg/kg), daily ×5
Gp 3: UTL-5g (60 mg/kg) followed by cisplatin (2.5 mg/kg) 30 min later, daily ×5
Gp 4: Cisplatin 0.4 mg/mL (5 mg/mL) in saline, day 1 only
Gp 5: UTL-5g (60 mg/kg) followed by cisplatin (5 mg/kg) 30 min later, day 1 only

Cisplatin was dissolved in saline. UTL-5g was prepared in DMSO/Cremophor/propylene glycol/saline as described in previous section. All injections were ip and the injection volume was 0.25 mL. Three days before treatment (Day −2), human colon cancer cells (HCT-15) were injected subcutaneously and bilaterally (1×10⁶ cells per site per mouse) in each SCID mouse in all groups and tumor sizes were measured by a caliper every 2-3 days and the animals were sacrificed when tumor reached 1,200 mm³.

The % T/C value is used here as the end-point. It is the ratio of (tumor size of treated group)/(tumor size of control) is an indication of the antitumor effectiveness. As shown in Table 1 below, when the mice were pre-treated with 60 mg/kg of UTL-5g before 0.2 mg/kg of cisplatin, the average % T/C (day 1-day 31) is 0.64 as compared to 0.82 for the group without the pretreatment of UTL-5g (Gp 3 vs Gp 2) indicating that UTL-5g actually increased the efficacy of cisplatin in this regimen. The enhancement was not observed for the single dose treatment with the average % T/C of 0.74 vs 0.76 for Gp 4 vs Gp 5. Since chemotherapy is usually given in a multi-treatment regimen, these unexpected results are unique and very encouraging.

TABLE 1
Average % T/C values of Gp 2-Gp 5 (n = 5)

| | Daily × 5 | | Single dose | |
|---|---|---|---|---|
| | Gp 2: | Gp 3: | Gp 4: | Gp 5: |
| | 0.2 mg/mL | UTL-5 g + 0.2 | Cisplatin 0.4 | UTL-5 g + 0.4 |
| Day | cisplatin | mg/mL cisplatin | mg/mL | mg/mL Cisplatin |
| −2 | Inj of | Inj of | Inj of | Inj of |
| | tumor cells | tumor cells | tumor cells | tumor cells |
| 1 | 1.00 | 1.00 | 1.00 | 1.00 |
| 3 | 0.81 | 0.44 | 0.84 | 0.81 |
| 5 | 0.82 | 0.75 | 0.82 | 0.75 |
| 8 | 0.63 | 0.46 | 0.47 | 0.44 |
| 10 | 0.80 | 0.59 | 0.82 | 0.70 |
| 12 | 0.66 | 0.49 | 0.73 | 0.51 |
| 15 | 0.84 | 0.63 | 0.85 | 0.88 |
| 17 | 0.84 | 0.65 | 0.77 | 0.86 |
| 19 | 0.78 | 0.69 | 0.77 | 0.85 |
| 23 | 0.91 | 0.66 | 0.75 | 0.78 |
| 24 | 0.87 | 0.65 | 0.71 | 0.80 |
| 26 | 0.84 | 0.66 | 0.67 | 0.81 |
| 29 | 0.80 | 0.64 | 0.57 | 0.73 |
| 31 | 0.87 | 0.70 | 0.65 | 0.77 |
| Avg | 0.82 | 0.64 | 0.74 | 0.76 |

To show the positive effect of UTL-5g, a representative plot for Gp 2 and Gp 3 vs. Control (Gp 1) is shown in FIG. 7.

Based on our studies, the important characteristics of UTL-5g can be summarized below:

1. UTL-5g lowered the elevated levels of blood urea nitrogen (BUN) and creatinine induced by cisplatin in vivo indicating the protection of kidney by UTL-5g.

2. UTL-5g lowered the elevated levels of blood aspartate aminotransferase (AST) and alanine aminotransferase (ALT) induced by cisplatin in vivo indicating the protection of liver by UTL-5 g.

3. UTL-5g achieved the same extent of chemoprotection in kidney and liver (as stated in #3 and #4) but at a much lower dose (60 mg/kg or 0.22 mmole/kg) as compared to amifostine (200 mg/kg or 0.93 mmole/kg). Therefore, UTL-5g is a more effective chemoprotector.

4. UTL-5g lowered the elevated levels of blood TNF-α induced by cisplatin in a dose dependent manner in vivo indicating that the protection of normal cells is related to the down regulation of TNF-α at least in part.

5. UTL-5g increased platelet count that was reduced by cisplatin in vivo; Amifostine does not have similar positive effect on platelet production.

6. UTL-5g by itself increased platelet counts in mice not treated by cisplatin indicating that UTL-5g stimulates the production of platelets.

7. UTL-5g did not have tumor-protective effect and actually increased the efficacy of cisplatin in vivo indicating that UTL-5g is a really unique compound that does not function according to conventional wisdom. On the other hand, amifostine was reported to have reduced efficacy on cisplatin. Animal data suggest that amifostine may have tumor-protective effect due to chemical quenching of cisplatin by the sulfur anion of the sulfhydryl moiety to form an inactive platinum-thiol conjugate species [Ethyol, 1996; Ethyol, 1998].

8. UTL-5g has a low acute toxicity in mice (LD₅₀>200 mg/kg) [Shaw, 2011].

Therefore, UTL-5g is a unique chemoprotective agent that not only protects liver, kidney, and platelets, but also increases the therapeutic effect of cisplatin simultaneously. Therefore, UTL-5g is a superior chemoprotective agent as compared to amifostine. In addition, UTL-5g by itself also stimulates the production of platelets. REPRESENTATIVE EXAMPLES OF RADIOPROTECTION BY UTL-5G

Similar to chemotherapy, radiotherapy also elevate TNF-α levels in surrounding tissues and blood. Inhibition of TNF-α pathway by an antisense oligonucleitide has been reported to be liver radioprotective [Huang, 2006]. However, there has been no report on the small molecule, UTL-5g, for its radioprotective effect.

### Example 5

### Radioprotective Effects of UTL-5b, -5d, and -5g

In a preliminary study, three UTL compounds (5b, 5d and 5g) were examined for their radioprotective effect in C57BL/6 mice. One hr prior to liver irradiation (15 Gy), animals were treated with the test compounds by i.p. injection (30 mg/kg). Two hr later, the animals were sacrificed and serum AST and ALT enzyme activities were determined. As shown in FIG. 8, pretreatment of the animals with these compounds lowered serum AST and ALT levels induced by irradiation. Although UTL-5d [Qian, 2005] and UTL-5b showed radioprotective effect in this preliminary study, UTL-5g was the most effective in lowering both AST and ALT enzyme activities (never public disclosed until now) and was selected for further experiments.

During the preliminary study, it was found unexpectedly that the vehicle used, DMSO/(Cremophor:propylene glycol 6:4)/saline 5:5:90 v/v was associated with some liver toxicity as indicated by the moderate increase of serum AST and ALT activities in animals treated with vehicle alone. Therefore, a modified vehicle, DMSO/EtOH/saline (5/5/90 v/v) was prepared and compared to that of vehicle I. Treatment of animals with the new vehicle showed essentially no toxicity (data not shown) as compared to saline control and was selected as the desired vehicle for preparing UTL-5g solution in subsequent studies.

### Example 6

### Dose-Dependent Radioprotective Effect of UTL-5g

Based on the preliminary studies, UTL-5g was selected for dose-dependent radioprotective studies. In this study, animals were treated with increasing doses of UTL-5g (from 0 to 60 mg/kg) one hr prior to liver irradiation (15 Gy). Two hr after irradiation, animals were sacrificed and serum AST/ALT enzyme activities and liver TNF-α levels were determined. As shown in FIG. 3, liver irradiation at 15 Gy significantly induced serum AST/ALT activities (increased from 52 to 135 Unit/L and 15 to 29 Unit/L, respectively). Pretreatment of UTL-5g one hr before irradiation significantly reduced serum AST/ALT activities in a drug dose-dependent manner (p<0.05, paired t test). For animals pre-treated with UTL-5g at 30 mg/kg, AST/ALT activity levels were lowered from 135/29 to 89/22 Unit/L, and those pre-treated with UTL-5g at 60 mg/kg, AST/ALT levels were reduced from 135/29 to 74/19 Unit/L, respectively. However, treatment with high dose of UTL-5g (60 mg/kg) alone slightly increased the levels of both AST and ALT enzyme activities in animals without irradiation (FIG. 9).

As shown in FIG. 10, TNF-α levels in liver extracts from irradiated mice (15 Gy) were increased by 56% compared with non-irradiated control animals (from 37.6 to 58.9 pg/mg tissue protein). Likewise, pre-treatment of animals with UTL-5g significantly reduced liver TNF-α levels by 20% and 29% at 30 mg/kg and 60 mg/kg, respectively.

### Example 7

### Radiation Doses and Radioprotective Effect of UTL-5g

Next, we investigated the radioprotective effect of UTL-5g with increased irradiation doses. In this study, animals were pre-treated with UTL-5g at 60 mg/kg and randomly divided into 4 groups. One hr later, they were subjected to liver irradiation at 0, 5, 15 and 25 Gy. Consistent with the previous study, pretreatment with UTL-5 g at 60 mg/kg was radioprotective against 15 Gy as evidenced by the reduction of serum AST activity (103.0 vs. 159.8 Unit/L in UTL-5g and in control vehicle groups, respectively) and ALT activity (17.8 vs. 32.3 Unit/L in UTL-5g and in control vehicle groups respectively) (FIG. 11). However, only moderate protection was observed in group of mice irradiated with 25 Gy. At lowest irradiation dose (5 Gy) used in this study, both AST and ALT levels were slightly but not significantly changed.

### Example 8

### TUNEL Assay for Apoptotic Liver Cells

To further analyze the radioprotective effect of UTL-5g, we examined the number of liver apoptotic cells by in situ TUNEL staining At the end of treatment, liver tissue sections were prepared, fixed and stained with TUNEL staining for apoptotic cells. There were “spontaneous” apoptotic liver cells in control animals as detected by TUNEL staining (20/five random fields), approximately 0.2% of total liver cells. The numbers of TUNEL positive cells in the tissue sections were markedly increased (82/five random fields) (>4-fold) by 15 Gy of radiation as compared to non-irradiated controls (FIG. 12). The radioprotective effect of UTL-5g was dose-dependent. At the lowest dose of UTL-5g (7.5 mg/kg), there was no significant protective effect. However, for higher doses (15, 30, and 60 mg/kg), the numbers of TUNEL positive cells induced by irradiation in the liver were significantly reduced from 4.1 to 3.3, 1.8, and 1.4-fold for animals treated with 15, 30, and 60 mg/kg UTL-5g, respectively. On the negative side, treatment with UTL-5g alone at the higher doses (30 and 60 mg/kg) also slightly increased the number of apoptotic cells in the liver tissue section from non-irradiated animals.

### DISCLOSURE OF THE PRESENT INVENTION

Disclosed in the present invention is a method of treating cancer patients with a small-molecule compound, UTL-5g, in combination with one or a plurality of other chemotherapeutic agents and/or radiotherapy so that the side effects chemotherapy or radiotherapy can be reduced, wherein the structure of compound UTL-5g is shown below:

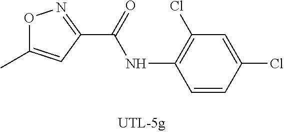

In the method, UTL-5g is administered to the patient before, during, or after the chemotherapeutic agent(s) and through one or a plurality of methods comprising oral administration, injection, implantation, topical application, and other suitable ways of administration for drugs, wherein suitable pharmaceutical excipients are used in the formulation of UTL-5g; said exicpients comprise one or a plurality of the following: water, saline, colloidal silicon dioxide, crospovidone, hypromellose, lactose monohydrate, magnesium stearate, polyethylene glycol, povidone, starch, talc, titanium dioxide, and suitable pharmaceutical coloring agent(s). Said side effects comprise damage to kidney, liver, and bone marrow; the damage of bone marrow comprises the reduction of platelet count. Said chemotherapeutic agents comprise cisplatin, carboplatin, oxaliplatin, satraplatin, and nedaplatin.

Also disclosed in the present invention is a method of treating thrombocytopenia patients with a small molecule UTL-5g, wherein the structure of compound UTL-5g is shown below

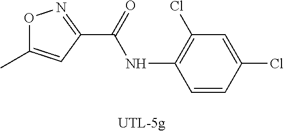

In this method, UTL-5g is administered through one or a plurality of methods comprising oral administration, injection, implantation, topical application, and other suitable ways of administration for drugs, wherein suitable pharmaceutical excipients are used in the formulation of UTL-5g; said exicpients comprise one or a plurality of the following: water, saline, colloidal silicon dioxide, crospovidone, hypromellose, lactose monohydrate, magnesium stearate, polyethylene glycol, povidone, starch, talc, titanium dioxide, and suitable pharmaceutical coloring agent(s). The thrombocytopenia patients comprise patients with acute leukemia, patients treated with chemotherapy, and patients treated with radiotherapy.

### SUMMARY, RAMIFICATION, AND SCOPE

In conclusion, novel methods of using UTL-5g for treating cancer patients and patients with thrombocytopenia are disclosed in this invention.

Although the description above contains many specificities, these should not be construed as limiting the scope of the invention but as merely providing the illustrations of some of the presently preferred embodiments of this invention. Thus the scope of this invention should be determined by the appended claims and their legal equivalents, rather than by the examples given.

1. A method of treating cancer patients with a compound, UTL-5g, in combination with (a) one or a plurality of other chemotherapeutic agents and/or (b) radiotherapy so that the side effects of chemotherapy and/or radiotherapy can be reduced, wherein the structure of compound UTL-5g is shown below:

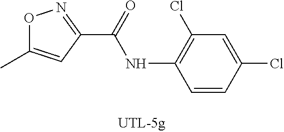

2. The method according to claim 1 wherein compound UTL-5g is administered to the patients before, during, or after the chemotherapeutic agent(s).

3. The method according to claim 1 where in compound UTL-5g is administered through one or a plurality of methods comprising oral administration, injection, implantation, topical application, and other suitable ways of administration for drugs.

4. The method according to claim 1 wherein suitable pharmaceutical excipients are used in the formulation of UTL-5g; said exicpients comprise one or a plurality of the following: water, saline, colloidal silicon dioxide, crospovidone, hypromellose, lactose monohydrate, magnesium stearate, polyethylene glycol, povidone, starch, talc, titanium dioxide, and suitable pharmaceutical coloring agent(s).

5. The method according to claim 1 wherein said side effects comprise damage to kidney, liver, and bone marrow.

6. The method according to claim 4 wherein the damage of bone marrow comprises the reduction of platelet count.

7. The method according to claim 1 wherein the chemotherapeutic agents comprise cisplatin, carboplatin, oxaliplatin, satraplatin, and nedaplatin.

8. A method of treating thrombocytopenia patients with a compound, UTL-5g, wherein the structure of UTL-5g is shown below:

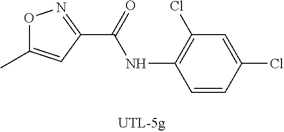

9. The method according to claim 7 wherein UTL-5g is administered through one or a plurality of methods comprising oral administration, injection, implantation, topical application, and other suitable ways of administration for drugs.

10. The method according to claim 7 wherein suitable pharmaceutical excipients are used in the formulation of UTL-5g; said exicpients comprise one or a plurality of the following: water, saline, colloidal silicon dioxide, crospovidone, hypromellose, lactose monohydrate, magnesium stearate, polyethylene glycol, povidone, starch, talc, titanium dioxide, and suitable pharmaceutical coloring agent(s).

11. The method according to claim 7 wherein thrombocytopenia patients comprise patients with acute leukemia, patients treated with chemotherapy, and patients treated with radiotherapy.
